(19) United States
(12) Patent Application Publication
(10) Pub. No.: US 2026/0248596 A1
(43) Pub. Date: Aug. 27, 2026

(54) IMMEDIATELY LOADED INTRAOSSEOUS SCREW IMPLANT AND ITS INSTALLATION METHOD

(71) Applicant: PLASMOVITA, Indianapolis, IN (US)

(72) Inventor: Vladimir Grigorevich Khantsis, Indianapolis, IN (US)

(21) Appl. No.: 18/856,199

(22) PCT Filed: Jun. 5, 2024
(86) PCT No.: PCT/RU2024/000177

§ 371 Date: Oct. 11, 2024

(30) Foreign Application Priority Data
Jun. 7, 2023 (RU) 2023114979

(51) Int. Cl. A61C 8/00

(52) U.S. Cl. CPC A61C 8/0025

(57) ABSTRACT

An intraosseous screw implant pertains to medical equipment. The device is made with a variable height of the single-thread profile (2). Within the full turns (8) of the screw line, projections (7) with an increased height of the thread profile (2) are localized. The projections (7) are intended to bear the immediate load of the orthopedic structure. The projections (7) are located on adjacent turns (8) and are mutually offset by an angle. In an alternative embodiment of the device, the projections (7) are located from each other through a turn (8). The method for installing the implant includes forming a bone bed in the form of a narrowing opening. The dimensions of the opening are selected based on the condition of free accommodation of the predominant part of the implant. The implant is inserted into the cavity of the bed with the projections (7) resting on the spongy bone layer. Then the implant is screwed into the bone with several turns for fixation. The shape of the implant is optimized based on the condition of intensification of osseointegration of the immediately loaded implant. Reduced traumatic effect of implant screw elements on bone while ensuring immediate biomechanical load. Compacted bone tissue particles in the chip groove (4) at the top of the implant. Increased reliability of osseointegration. Increased implant survival level.

### TECHNICAL FIELD

The invention relates to medical equipment, namely to endosteal supporting structures of the screw type, intended for immediate loading after their implantation. The preferred areas of application of the technical solution are orthopedic dentistry and orthopedics of the musculoskeletal system.

### PRIOR TECHNICAL LEVEL

For immediate loading of a screw implant, its high primary biomechanical stability in the bone is required, which is ensured by the structural elements of the device. However, these elements can additionally injure the bone and subsequently lead to a violation of the osseointegration process, due to which the percentage of rejections increases in practice.

From patent document U.S. Pat. No. 8,932,059 B2 dated 13 Jan. 2015, a dental screw implant is known, made with a variable profile height of the turns due to the different height of the threads of the two-way thread. A thread with a higher profile height of the turns serves to provide the implant with primary stability, and a thread of a smaller size is intended to increase the contact area of the implant with the bone, which has a positive effect on the process of further osseointegration. The size of the thread turns specified in the known design, providing primary stability of the implant, requires a force for screwing in the device that can cause traumatic stress of the bone and compression necrosis with subsequent resorption, and ultimately rejection of the implant.

The risk of rejection is high due to the occurrence of bone tissue necrosis along the entire length of the implant in the area of the thread apex with a greater thread profile height. This creates a risk of focal necrosis areas joining, which reduces the blood supply to the damaged area and slows down tissue reparation. Due to the large thread pitch that provides primary stability of the known implant, the load on the bone surrounding the implant is so uneven that foci of bone tissue necrosis may appear in the immediate vicinity of the thread surface with a greater thread profile height. However, alternating threads with different thread profile heights does not allow for the production of an implant with a smaller thread pitch, since this will violate the target properties of the threads of the other thread. In addition, the pitch angle of the two-way thread is too high to exclude the risk of spontaneous rotation of the implant under the action of axial forces. Chip grooves with cutting edges are formed on the body of the known implant. The screw shape of the grooves ensures that bone particles are diverted from the head of the implant body to its middle part during the installation of the device. In addition, the grooves stabilize the implant and give it anti-rotational properties. However, the bone mass in the grooves is greatly loosened, which increases the duration of the recovery process and reduces the effectiveness of the specified properties of the grooves.

Also, a method for installing an intraosseous screw implant is known from the mentioned patent document, made with a variable height of the profile of the turns, the overall shape of the body of which in the middle and neck parts is characterized by a narrowing towards the apex. According to the known method, a bone bed for the implant is first formed, having the form of an opening narrowed towards the bottom. Then the implant is sequentially screwed into the cortical and spongy layers of the bone for fixation. However, wherein the thread turns with a greater profile height pass through the hole from the bone surface to the bottom of the bed, due to which they have a multiple traumatic effect on the bone tissue with each revolution of the implant body.

### DISCLOSURE OF THE INVENTION

The technical problem is to create objects of the invention that are devoid of the disadvantages of the known device and method, including optimization of the shape of the screw implant from the condition of intensification of the process of osseointegration of the immediately loaded implant, increasing the reliability of such osseointegration, and also increasing the survival of the said implant.

The positive effects provided by the invention consist, in relation to the device and method known from the prior art, firstly, in reducing the traumatic effect on the bone of the screw elements of the implant while ensuring that they bear the immediate biomechanical load applied to the implant, and secondly, in increasing the density of bone tissue particles in the chip groove on the top of the implant.

The above is achieved due to the fact that the intraosseous screw implant, made with a variable thread profile height, contains such turns of the helical line that within each of them at least one projection with an increased thread profile height is localized, having a shorter length compared to the length of the thread turn. In this case, the above projections are located on adjacent turns and are mutually offset by an angle. In an alternative embodiment of the device, the projections are located from each other through a turn.

In a particular case of the invention, the projections are formed in the middle part of the implant body and are placed on its surface in a checkerboard pattern.

In another particular case, straight chip grooves are made on the top of the implant body, turning into helical chip grooves in the middle part of the implant body. In this case, the said grooves divide the thread to form rows of blades, and along the thread, blades with different profile heights alternate.

In yet another particular case, blades with different profile heights alternate in each row.

Also, the technical problem is solved, and positive effects are ensured, due to the fact that for installation of an intraosseous screw implant, the overall shape of the body of which in the middle and neck parts is characterized by narrowing towards the apex, containing such full turns of the helical line that within each of them at least one protrusion with an increased height of the thread profile is localized, first a bone bed is formed in the form of a narrowing hole, the dimensions of which are selected from the condition of free accommodation of the predominant part of the implant with an emphasis on the spongy layer of the bone of the protrusions with an increased height of the thread profile. Then the implant is inserted into the cavity of the bed until it stops, and then screwed into the bone for fixation. The traumatic effect on the bone of the screw elements of the implant while ensuring the bearing of an immediate biomechanical load is reduced due to a decrease in the size of the thread turns bearing the immediate load. At the same time, the value of the primary stability of the implant remains unchanged due to the increase in the number of thread elements bearing the immediate load, which allows maintaining a sufficiently large value of the working surface area of the thread, ensuring the primary stability of the implant.

The increase in the number of said elements is ensured by combining sections within the turns both for maximizing the primary stabilization when applying an immediate load to the device after implantation and for minimizing the time of tissue healing and osseointegration of the implant, for which purpose the thread contains at least two full turns, within each of which at least one protrusion with an increased thread profile height is localized. Also, a decrease in the size of the thread turns bearing the immediate load is ensured by a more uniform distribution of the biomechanical load on the bone surrounding the implant. Uniformity of load distribution is achieved by making the said protrusions on adjacent thread turns with a mutual offset by angle or through a turn, which ensures that different types of retention elements of the implant are distanced from each other. To increase the uniformity of load distribution on the tubular bone layer, it is advisable to make the protrusions on the thread in the middle part of the implant body and place them on its surface in a checkerboard pattern. When the thread is made in the form of rows of blades, the increase in the uniformity of the load distribution is preferably ensured by alternating blades with different profile heights along the thread. If the blades with different profile heights also alternate in each specified row, then the value of the uniformity of the load distribution is maximum, which makes it possible to optimize the sizes of the thread protrusions. The spacing of the thread protrusions prevents the risk of merging focal areas of necrosis and creates conditions for increasing the blood supply to the damaged areas and rapid reparation of bone tissue, since when necrosis occurs, its zones are localized by thread protrusions and are shifted relative to each other, due to which the blood supply to limited necrotic areas is increased, which leads to acceleration of osseointegration and reduces the risk of implant rejection.

In addition, the traumatic effect of the screw elements of the implant on the bone while ensuring immediate biomechanical load bearing is reduced due to the implementation of the implant with a narrowing towards the top of the overall shape of its body in the middle and neck parts. To install this implant, the bone bed is formed in the form of a narrowing hole, the dimensions of which are selected based on the condition of free accommodation of the predominant part of the implant with an emphasis on the spongy layer of the bone of the protrusions with an increased height of the thread profile, which makes it possible to avoid multiple traumatic effects on the bone tissue with each turn of the implant body when screwing the implant into the bone for its fixation, since for reliable fixation of the implant in the bone, only a few full turns of the implant around the longitudinal axis are sufficient without creating undesirable overheating of the bone, leading to tissue necrosis. In this case, the protrusions with an increased height of the thread profile do not pass through the cortical part of the bone and do not damage this layer, in which only the microthread on the neck of the implant body is fixed. The implementation of the implant body with chip grooves ensures the collection and accumulation of bone tissue particles during the installation of the device, and during the osseointegration process, it imparts stability and anti-rotational properties to the implant. Moreover, due to the straight chip grooves, the density of bone tissue particles at the top of the implant is increased due to the compression of these particles, which leads to an acceleration of the bone tissue restoration process in this area and the prompt manifestation of the implant stability and its anti-rotational properties, since the resulting bone tissue stops the implant. In addition, due to the chip grooves, which occupy the entire length of the implant body, multiple cutting edges of the thread blades are formed, due to which, when installing the implant, it is not necessary to apply a large force with traumatic consequences.

The screw chip grooves also provide increased torsional mechanical rigidity of the implant body, which has a positive effect on further osseointegration.

The connection of the implant to the bone has sufficient strength, including due to the implementation of the implant body with a single-thread thread, which reduces the risk of unwanted rotations of the endosteal part of the implant under the action of axial forces applied to the prosthesis.

### DESCRIPTION OF THE DRAWINGS

The essence of the invention is explained by the following drawings, which show two basic designs of a dental root-shaped implant as preferred examples of implementing the technical solution.

FIG. 1-2: general view of the implant and cross-sections of its body.

FIG. 3-4: alternative implementation of the implant, general view and cross-sections.

FIG. 5-6: schematic image on the cross-section of the implant of one and two protrusions with an increased height of the thread profile.

### THE BEST EMBODIMENT OF THE INVENTION

The presented technical solution is a support-retaining endosteal part of a device intended for single-stage dental implantation with immediate loading by an orthopedic structure.

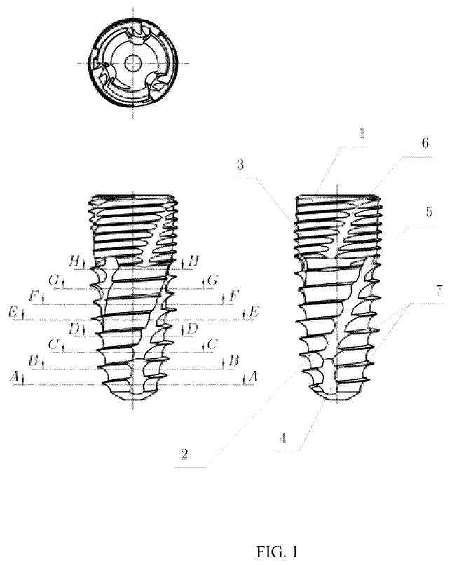

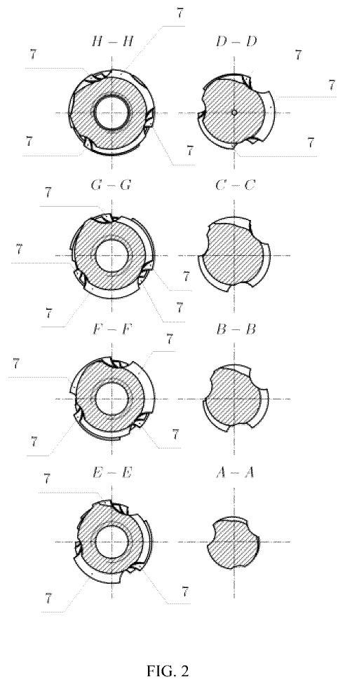

On the lateral surface of the implant body 1, retention elements are made in the form of an external screw thread 2 in the middle and apical parts of the body 1 (FIGS. 1 and 2), ensuring the possibility of primary and secondary stability of the implant in the spongy bone layer. On the neck of the body 1, a retention screw microthread 3 is cut for fixing the implant in a thick and dense cortical bone layer.

Three open peripheral grooves are made on body 1, each of which consists of interconnected chip grooves 4 and 5, dividing the thread 2 into longitudinal rows of blades in the form of separate segments of an intermittent thread with cutting edges. In each row, the blades are shifted from each other by an axial angle. Chip grooves 4 on the top of body 1 are straight, since they are located parallel to the longitudinal axis of this body. The chip grooves 5 in the middle part of the body 1 have a helical configuration and serve as a continuation of the straight chip grooves 4. Three separate helical chip grooves 6 are formed on the neck of the body 1.

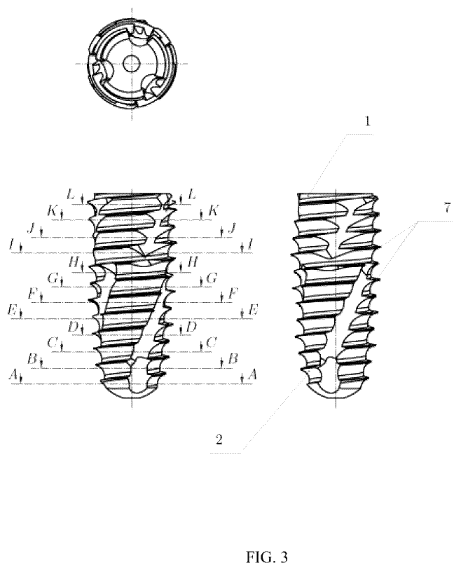

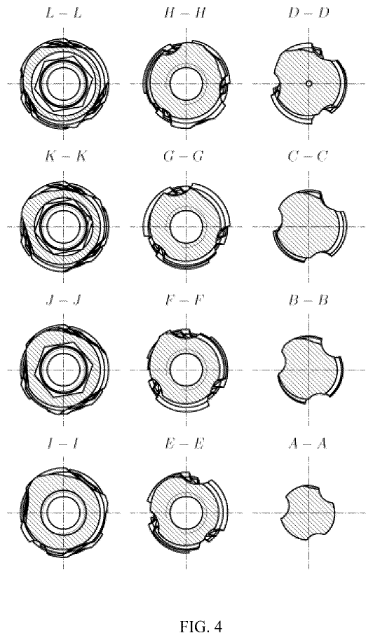

The thread 2 is made single-pass and consists of a single thread with a small angle of inclination of the turns, sufficient to form 7-10 full turns of the thread 2 in the middle and apical parts of the body 1 of the implant. If, instead of a microthread 3, a thread 2 is cut on the neck of the implant (FIGS. 3 and 4), which is permissible in the case of a thin cortical layer, then the number of turns of the thread 2 exceeds the specified values.

The helical line of the thread 2 is characterized by a variable height of its profile due to the fact that within the full turns of the helical line in the middle and apical parts of the body 1, projections 7 of the thread 2 are localized, each of which represents a protruding part of the body 1, limited by the helical surface of the thread 2 and having the form of a blade with an increased height of the profile of the thread 2 (FIGS. 2 and 4). The microthread 3 is made without projections. The geometric parameters of the elements of the thread 2 are selected from the following system of independent inequalities.

$$\{ \begin{matrix}{W}_{1} > {W}_{0} \\ α < 360 ° \\ β > 0 °\end{matrix}$$

Where:
W0—base height of the thread profile;
W1—height of the thread projection profile;
α—angular length of the thread projection;
β—mutual angular displacement of the thread projections.

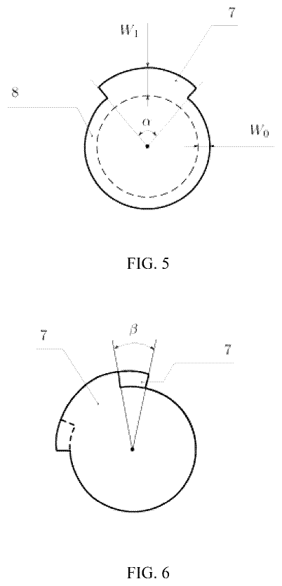

Each projection 7 is characterized by a greater profile height W1 of the thread 2 relative to the base height of the profile W0 of this thread. The base height W0 is selected based on the condition of minimizing the time of tissue healing and osseointegration of the implant, and the height W1 of the projections 7 is selected based on the condition of ensuring the maximum possible primary stabilization when applying an immediate load to the device after implantation. The angular length a of the projection 7 has a smaller value than the angular length of a full turn 8 of the thread 2 (FIG. 5), which is 360°. Moreover, the edges of the projections 7 on the adjacent turns 8 are shifted relative to each other by an angle β (FIG. 6). Blades with different heights of turns 8 alternate along the thread 2. Since the device contains an odd number of rows of blades, they alternate in each row. In an alternative embodiment of the device, projections 7 are distributed over the surface of the middle part of the implant in a checkerboard pattern.

The top of the body 1 is rounded. The overall shape of the body 1 in the middle and neck parts is characterized by a weakly expressed narrowing towards the top. In general, the body 1 has a circular cross-section.

The body of the implant 1 is made of titanium alloy, zirconium dioxide or a similar medical material with suitable properties.

The installation of the screw implant in the bone is performed in one stage as follows.

First, a bone bed is formed in the form of a tapering blind hole using a conical or step drill. The drill size is selected based on the condition that it corresponds to the overall shape of the implant, so that it is possible to freely insert the body of the implant 1 into the prepared hole approximately two-thirds the length of this body. Then the implant is inserted into the cavity of the bed to the specified depth with the projections 7 resting mainly on the spongy bone layer, the micro thread 3 is located in the immediate vicinity of the cortical bone layer. To ensure the primary stability of the implant, the body 1 is screwed into the bone by 2-3 full turns. In this case, the cutting edges of the blades form particles of the spongy bone layer, which are first compacted by the walls of the straight chip grooves 4, and then moved to the middle part of the body 1 by the screw chip grooves 5. The particles of the cortical layer are removed by the grooves 6. After the fixation of the implant body 1 in the bone is completed, an abutment and a dental prosthesis are attached to the implant. Over time, osteoclastic processes occur at the implantation site, leading to the removal of bone tissue damaged by the screw blades of the body 1, due to which the primary stability of the implant gradually decreases. However, secondary stability increases due to the development of osseointegration, which fully covers first the compressed bone particles in the chip grooves 4, and then the particles in the grooves 5, 6. The said optimization of the shape of the screw implant, not limited to the best embodiment of the invention, leads to an intensification of the osseointegration process of the immediately loaded implant, an increase in the reliability of such osseointegration, and as a result, to an increase in the survival rate of the implant.

1. An intraosseous screw implant made with a variable thread profile height (2), characterized in that it contains such turns (8) of the helical line that within each of them at least one projection (7) with an increased thread profile height (2) is localized, having a shorter length compared to the length of the turn (8) of the thread (2), wherein said projections (7) are located on adjacent turns (8) and are mutually offset by an angle or are located from each other through a turn (8).

2. The implant according to claim 1, characterized in that the projections (7) are formed in the middle part of the implant body (1) and are placed on its surface in a checkerboard pattern.

3. The implant according to claim 1, characterized in that its apex has straight chip grooves (4) that transition into helical chip grooves (5) in the middle part of the implant body (1), wherein said grooves divide the thread (2) to form rows of blades, wherein along the thread (2) the blades with different profile heights alternate.

4. The implant according to claim 3, characterized in that the blades with different profile heights alternate in each row.

5. A method for installing an intraosseous screw implant, the overall shape of the body (1) of which in the middle and neck parts is characterized by a narrowing towards the top, containing such turns (8) of the helical line that within each of them at least one protrusion (7) with an increased height of the thread profile (2) is localized, including the formation of a bone bed in the form of a tapering opening, the dimensions of which are selected from the condition of free accommodation of the predominant part of the implant with an emphasis on the spongy layer of the bone of the protrusions with an increased height of the thread profile (2), after which the implant is inserted into the cavity of the bed until it stops, and then screwed into the bone for fixation.
